(19) United States
(12) Patent Application Publication
(10) Pub. No.: US 2024/0011966 A1
(43) Pub. Date: Jan. 11, 2024

(54) WIRELESS SOIL TESTER WITH REAL-TIME OUTPUT

(71) Applicants: BRIAN BOSIRE, NAIROBI (KE); EVAN WADONGO, NAIROBI (KE)

(72) Inventors: BRIAN BOSIRE, NAIROBI (KE); EVAN WADONGO, NAIROBI (KE)

(21) Appl. No.: 18/026,795

(22) PCT Filed: Aug. 19, 2021
(86) PCT No.: PCT/KE2021/050005

§ 371 Date: Mar. 16, 2023

(30) Foreign Application Priority Data
Aug. 19, 2020 (KE) KE/P/2020/003709

(51) Int. Cl. G01N 33/24; A01B 79/00; G16Y 20/10; G16Y 40/10; G16Y 10/05

(52) U.S. Cl. CPC G01N 33/246; A01B 79/005; G16Y 20/10; G16Y 40/10; G16Y 10/05; G01N 2033/245

(57) ABSTRACT

The disclosure relates to agricultural sensors for precision farming. It tests for soil moisture content, Potential of Hydrogen (pH), salinity, nutritional content, farm topology and employs the Internet of Things (IoT) technologies to analyze the data and make a prescription in real time. The disclosure is a combination of a handheld portable device system and a cloud-based analytics Platform. The hand-held device system contains a micro-processor, memory, keypad input interfaced with the output via a display, signal conditioner, data encoder and decoder, local bus allowing for in system communication, communications module with wireless communications capability and power management module. The hand-held device has attached probes for manual insertion into the soil for testing. The cloud-based platform consists of a database, algorithm and Application Programming Interface (API) which receives device data for processing. The derived recommendations are routed to an output gateway and in readable text format.

### DETAILED DESCRIPTION OF THE INVENTION AND DRAWINGS

### Technical Field

The disclosure relates to agricultural sensors for precision farming applications, including sensors configured to measure soil, farm location and conditions in real-time and to send associated data via a communication network for example GSM 2G or 3G network to cloud server hosted database and data analytics and processing platform.

Background Art

The cloud connected sensor devices using Internet of things (IoT) technologies are presently used to build decision support systems relying on field sensors that collect data to overcome many problems in the real-world. Precision agriculture (PA) is in increasing need of decision support systems especially in the context of largescale farms. Smallholder farmers have been left out of these technologies due to high cost of adoption and challenges in making use of the collected data to make knowledge driven decisions in crop production. This disclosure presents a means to solve the agricultural problems related to farming resources optimization at farm level, decision making support, and farm and soil monitoring. This approach provides real-time information based on the farm specific conditions and crops that will help farmers make right decisions using principles of IoT, sensor and data analytics technologies.

Lack of data and information for small scale farmers has resulted in farmers making uninformed farming decisions, for example, regarding choice of fertilizers, seeds and other decisions related to producing a robust harvest. Unreliable information on weather has in additionally contributed to losses due to the climate change. While the existing soil sensors are designed to sense the soil moisture content in situ by burying and leaving them in the soil, the other soil variables such as soil nutrients, soil acidity and salinity which are essential for precision farming are left out by these sensors. Additionally, burying the soil sensors limits reuse and sharing of the sensors amongst different users in different farm locations, considering the high costs of purchasing and installation of such sensors. This makes them unaffordable and out of reach of the smallholder farmers leading to limited adoption of the technology and locking these farmers out of precision farming capabilities. Commercial sensors attached to tractors and farm vehicles are also limited to large scale farms leaving out the small farm owners.

### DISCLOSURE OF INVENTION

This disclosure presents new and useful improvements in soil testers and sensors. This approach using principles of IoT, sensor and data analytics technologies provides real-time information based on the farm specific conditions that will help farmers make right decisions.

The disclosure encompasses an electronic sensor device disclosed in FIG. 3 consisting a hand-held unit connected to the sensory unit by a code, 304, to transfer the sensor data to the handheld unit comprising of components disclosed in FIG. 1, that communicates with a cloud-based platform whose functionality is illustrated by FIG. 4.

The device the system contains a micro-processor control unit, 102, an EEPROM memory unit, 106, having firmware stored therein that defines the abovementioned functionality, input and output (I/O) interfaces consisting of a display unit, 108, an Alphanumeric input, 114, Signal conditioning unit, 110, data encoder and decoder unit, 118, and a local bus, 120, allowing for communication within the system and a communications module, 104, with a wireless communications capability for example using standard GSM 2G, 3G network connection. The device is expandable to receive additional sensors.

The cloud-based platform consists of a comprehensive database and an Application programming interface (API) which may be built up on the standard programming languages or a combination thereof to implement methods for mathematical and probability formula for example the Bayesian inferencing to generate recommendations based on cloud-based database standard data and sensor data.

### BRIEF DESCRIPTION OF DRAWINGS

The accompanying drawings illustrate embodiments of the invention and, together with the description, serve to explain the invention.

FIG. 1 is a schematic diagram of the major internal components of the embodiment of FIG. 3 with illustration of data flow between the components of the embodiments of FIG. 3.

FIG. 2 is a flow diagram of an exemplary embodiment of a method for using the sensor device embodiment of FIG. 3. illustrating an example of a sensor device system executing functionality of the present invention

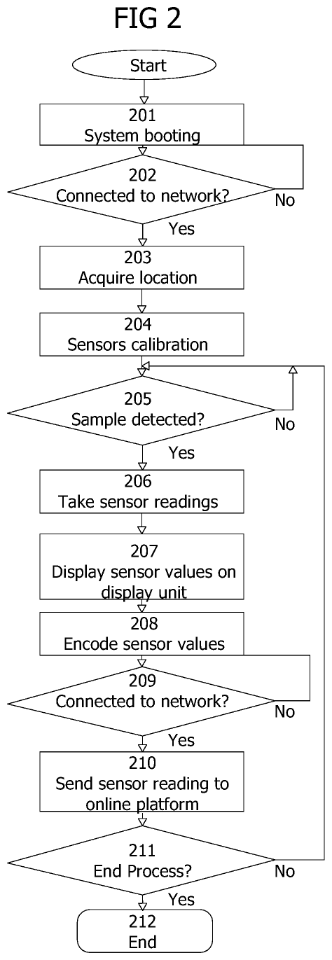

FIG. 3 is a diagram illustrating external components of an exemplary embodiment of a sensor device.

FIG. 4 illustrates an overview of a four- fold architecture of the system of the cloud-based platform in accordance with the present invention and depicting the different input datasets of through user interfaces and sensor data, the centralized database, recommender engine logic and data outputs in actionable format.

### BEST MODE FOR CARRYING OUT THE INVENTION

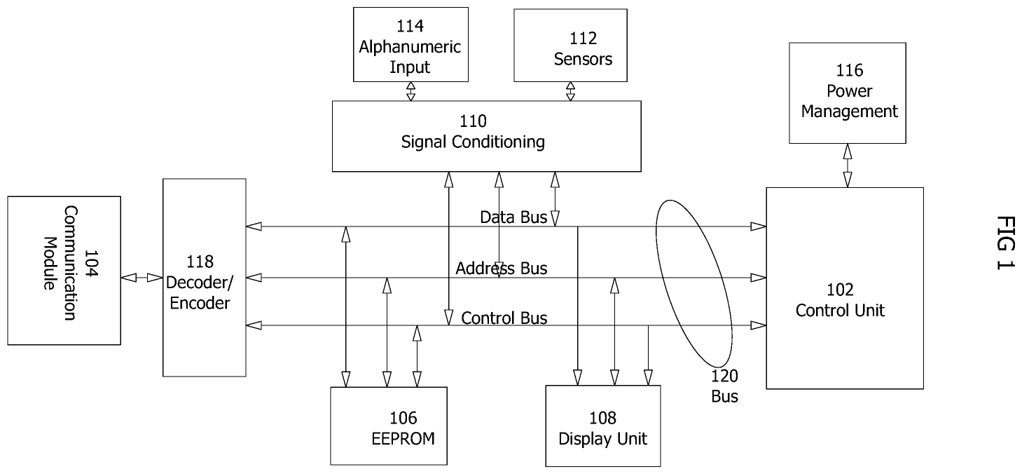

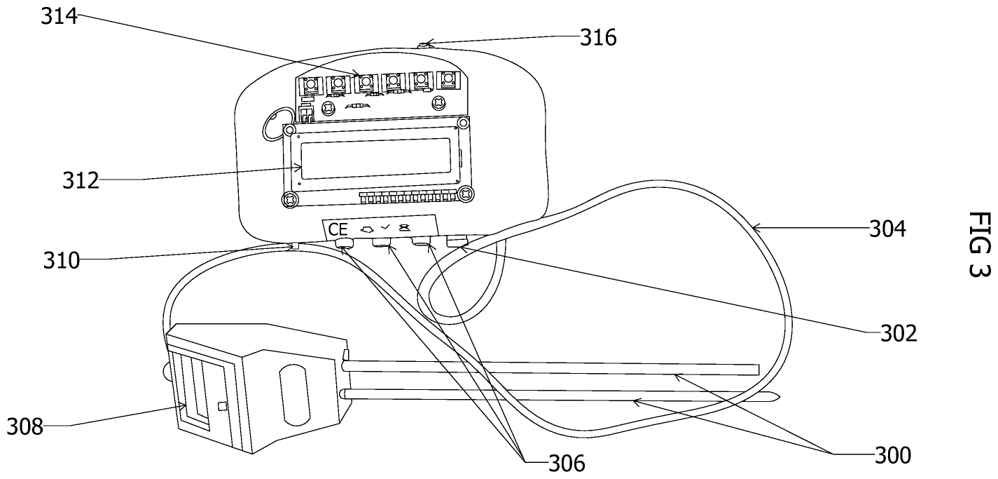

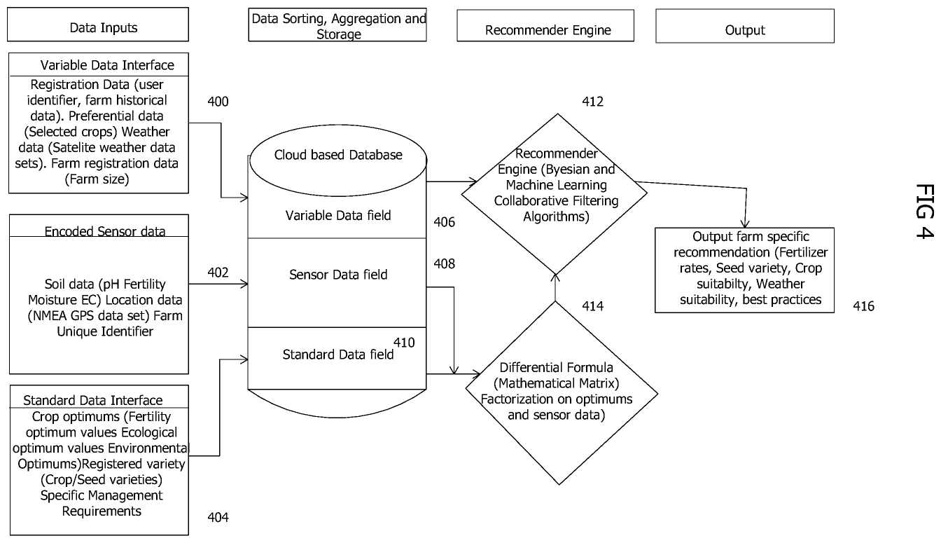

The solution encompasses an electronic sensor device as disclosed through external embodiments in FIG. 3 consisting a hand-held unit connected to the sensory unit by a code, 304, to transfer the sensor data to the handheld unit comprising of components disclosed in FIG. 1, that communicates with a cloud-based platform whose functionality is illustrated by FIG. 4. The sensor device is a soil and farm analysis device that is portable and handheld unit connected via a flexible cabling to a sensory module. The handheld unit houses an interactive interface through keypad 314 and display unit 312 and the sensory unit consisting of an integrated antimony-copper junction probe and aluminium-antimony junction probe pair to make up the Potential of hydrogen (pH) sensor, 300, Electrical Conductivity (EC) probe ,300, and Moisture sensor 300, Colorimetry sensor, Temperature and Infrared sensor 306. Unlike the previous solutions described in the background section the sensor device uses antimony-copper junction potential drop directly related to Potential of Hydrogen Ion (pH) in the measured sample formulated through the regression equation; pH=(x31 0.049)/0.056; for x being the Potential difference (PD) obtained from the sensor. The Electrical conductivity (EC) calculated from the measurement of sample Resistivity (R) values through constant current source across the Copper-aluminium pair, to obtain the inverse function in micro-siemens given by the formula; EC (Us)=(y(t)/(x(t)*0.0040155718))*1000000 where y(t) is the instantaneous input current value in Amperes (A) while x(t)=obtained PD value at that instant in Volts (V) and Volumetric Moisture inferenced through the sample resistivity(R) while the hand held unit houses GPS unit, 316 in the communication module, 104 elaborated in FIG. 1; Colorimetry and infrared unit 306 as in FIG. 3. Connected together, the sensory unit and handheld unit through flexible data cable 304, derive one or more indications of soil and/or environmental conditions, for example, the nutrient levels with the soil pH scale value ranging 3 to 10 sensed by the pH sensor through an antimony-copper junction; The available soil Nitrogen percentage value calculated from colorimetric sensor values through regression modelling using the function:
%N=antilog (8.925-10-0.1VC)=antilog (0.925-0.01VC)/100 V=
Mansell's colorimetric value (V)
C=Chroma [Chroma, C(a*b*)={a*\hat{ }2+b*\hat{ }2)}\hat{ }0.5];

Organic matter (OM) obtained through regression modelling using colorimetric sensor values by Calculation formula:
y=−3.088 ln(x)+9.228
x=Mansell's colorimetric value (V) and the y=%Soil Organic Carbon (SOC) Organic
Matter (OM)=Carbon (%) x 1.724;

The inferenced Phosphate value in parts per million (PPM) through predictive correlation factor modelling from the colorimetric sensor values converted into the Mansell's color system for sample image processing, inferenced Potassium levels in parts per million (PPM), Electrical Conductivity (EC) in micro-Siemens (uS) sensed through inverse resistivity calculation of the sample by a constant alternating current source through the probe sensor, 300, into the soil sample; and farm ambient conditions consisting of ambient temperature through the inbuilt sensor, GPS coordinates made up of encoded National Marine Electronics Associations (NMEA) 2000 standard format consisting of Longitude, Latitude and altitude, and encode the values to a string which is sent over a network such as GSM to the cloud based platform. The data is analysed through the process illustrated in FIG. 4 to generate recommendations which is made up of information with actionable data for example fertilizer application rate in Kilograms, suitable seed by variety, lime application rates in Tonnes per Acre all generated from a data analytics platform that receives the sensor data from the sensor device over a communications network such as GSM or 3G network through an Application Programming Interface (API) and uses an algorithm, 414, to compare the measured values and the crop optimum values, 410, keyed in as standard data through an Application interface, 404, in the database, 410, as in FIG. 4. The generated recommendation through collaborative filtering in the recommender engine, 412, is the is passed to through the output gateway API and sent as a message in a readable format, 416, over network such as GSM 3G for example through either email, SMS or mobile application as per to the preferential data keyed in as the variable data interface, 400, to the farmer's communications device. The sensor device using a wireless cellular network such as GSM 2G or 3G network sends alphanumeric encoded data with each dataset from each sensor separated by a symbol such as an asterisk, stroke or hash combined into a single string of characters that is then sent to the cloud based comprehensive database that receives the data string through an application interface, 402, which decodes the data and assigns each sensor value to the corresponding fields within the database, 408, for processing. The database, 406, 408, 410, contains standard agricultural pre-keyed in through an application interface, 404, such as climatic, soil and crop information that is relevant to the farming process stages. The database built on a scalable data management technology for example Structured Query Language (SQL) or MYSQL contains tables and fields, 406, 408, 410, of information that works as the container for the standard, variable and sensor data with crop optimum conditions based on the type of crop, variety of the crop with specific data on ecological requirements of the crop for example, altitude, soils, drainage, climatic requirements such as humidity or rainfall, temperature, market and consumption information. Individual farmer and farm details for example a unique farmer identifier matched with farm identifier matched soil data and farm location collected by sensor device, Availability of the seeds and input stores such as agro input dealers, regional information such as location for example counties or village of particular farm and farmers, suppliers and their locations. The data on the database is organized into specific fields to allow ease of processing by the APIs that use data analytics frameworks such as Hadoop and MapReduce to analyse the datasets and create a matching recommendation through the recommender engine, 412, 414, based on the farm's location derived from GPS co-ordinates from sensor data, farmer's history from the farmer's registration details in the database through variable data interface, 400, soil fertility data as derived from the sensor device, 402, preferred crops derived from variable data interface, 400, crop optimum information from the standard crop optimum requirements and seed availability from the distributor network data field. Recommendation consists of actionable easy to understand data processed by the machine learning API algorithm based on fuzzy logic and Bayesian models and prediction, machine learning which gives a matching output of the most suitable type of crop, variety of seed, options on where to get the seed within the nearest locality standard crop management information such as crop pruning requirements, weeding, fertilizer topdressing, pests and diseases. The data from the sensor device in the database is pulled by a software API which processes and outputs the nutrient requirements of a preferred crop by matching and calculating the difference in actual soil conditions and the optimum crop nutritional requirements based on the standard database crop data to output a recommendation, 416, of input use for example the calculated fertilizer rate, lime or sulphur rate for pH correction and fertilizer and manure ratios using standard mathematical formula that make up the processing API algorithm,414. The platform has APIs that mine data from existing online satellite weather data that are pulled as an encoded string format for example a JavaScript Object Notation (JSON) framework. This data sorted in fields in the database for example field of user such as a farmer registration details such as a unique identifier, crop variety data for example variety of a particular seed optimized for an ecological zone, with specific moisture and pH requirements for example, is mapped to the specific farm GPS co-ordinates which then return the expected weather conditions such as the locations' rainfall, temperature, humidity, wind strength for a predicted timeline for example daily prediction, weekly prediction and monthly prediction. This weather data is combined into a message string by an API bot then released sent to the farmers' communication device via a GSM network for example 2G or 3G in a format that is easily accessible for example a Short Message Service SMS or email. The sensor device connects to the cloud-based platform via the elaborated wireless cellular network to send the collected data sets and values to the database which uses an algorithmic processes described in FIG. 4 through sensor data Application interface, 402, to which undergoes processing as in FIG. 4 to provide an output which is sent as a message to the farmer's communications device, the message contains precise actionable information from the results of the soil and farm analysis data, providing easy to understand recommendations on required fertilizer, required soil treatment and provides information on certified seeds, certified distributors and where the farmer can get these inputs within his/her locality and other agronomical support tips all of which are fields and tables of the database hosted in the cloud platform. The handheld sensor device may operate like any other personal computerized gadget, like a mobile phone, always maintaining contact with the cloud-based platform through a network such as GSM 2G or 3G networks. Using the soil data, farmer data and location special data of the farm collected from the sensor device, the farmer may receive key messages on a scheduled timeline such as daily, bi weekly or a weekly basis with information such as predicted weather expected in the specific farm locations and market trends for the selected and cultivated crops that will enable him/her make informed farming decisions. Users, for example, farmers, may use the exemplary device, for example, during land preparation, prior to planting and at any time prior to applying any fertilizers to their farms. The sensor device may be used for farmers who want to practice intercropping and cultivate new farm lands, based on the data from the sensor devices; the users for example farmers may receive recommendations for example on the best suited or supported crop types in their farms based on for example the geographical location weather and soil characteristics at the farm. As illustrated in FIG. 4, The cloud-based platform, as disclosed herein, which may be hosted on for example a platform as a service PAAS provider for example cloud service providers or virtual servers having communications with the cloud-based platform. The platform has a comprehensive database container, 406,408,410, of up to date farming information for example crop varieties, ecological zones, and weather, with an API algorithm that uses Artificial Intelligence which may be built on top of existing machine learning and artificial intelligence infrastructure technologies to automate dissemination of agricultural information to farmers via wireless network such as GSM 2G or 3G to the farmer's communications device for example mobile phone. The farmers have the ability to query or send information using their communications device for example a mobile phone to a dedicated communications channel over for GSM 2G or 3G network for example, via a gateway for example a Short Message Service (SMS) Application Programming Interface (API) or e-mail gateway API that sends the message to the cloud-based database on the cloud platform. The received message is processed by an API that synthesizes the message using Artificial intelligence and machine learning and predictive modelling for example Bayesian modelling or Collaborative filtering algorithms to generate matching answers from a pool of data mined from database sources such as the specified crop optimums, specific crop best management practices for example pest management information matched with the particular crop which are pre-keyed into the database as standard data, the matching answers are forwarded to an interface where an expert evaluates and verifies the answer via a web application built on content management system (CMS) and web applications tools for example JavaScript, Hyper Text Mark-up Language (HTML) and Hypertext Processor (PHP) to create an interactive interface before okaying the response to be sent to the users thus allowing two way communications from the platform and users for example farmers over a communications device.

The sensor device may be expandable to receive additional sensors; for example, using additional ports that may be used through a firmware update which may be done either remotely through a wireless GSM network such as 2G or 3G, and then remotely triggered for an automatic upgrade of the firmware. The platform consisting of the database, the machine learning APIs, Artificial Intelligence engine and the Gateways are linked to operate as a unit as in FIG. 4, which allow the farm specific data collected from the sensor device and the gathered database content with user profiles, farm and their ecological profiles, market data, crop data and input distribution network data may be used for big data analytics, which may generate data driven insights, trends and predictions using mathematical models with regard to decision making processes by sector players for example fertilizer manufacturers to know the fertilizer blends suited for different farmlands over a wide area and optimize distribution, to monitor soil degradation, seed companies to optimize distribution, the policy makers on crop productivity research, market players on production trends and governments on prioritizing agricultural investments in a jurisdiction.

### The device System

As previously mentioned, the present system for executing the functionality described in detail above may be a handheld computerized unit, an example of which is shown in the schematic diagram of FIG. 1. The system contains a micro-processor control unit, 102, an EEPROM memory unit, 106, having firmware stored therein that defines the abovementioned functionality, input and output (I/O) interfaces consisting of a display unit, 108, an Alphanumeric input, 114, Signal conditioning unit, 110, data encoder and decoder unit, 118, and a local bus, 120, allowing for communication within the system and a communications module, 104, with a wireless communications capability for example using standard GSM 2G, 3G network connection via for example a Subscriber Identity Module (SIM) card, The local interface, 120, can be, for example but not limited to, one or more buses or other wired or wireless connections, as is known in the art and a power management module, 116, that controls the use of battery power, charging control and supply to the various units of the embodiment.

### The Cloud based Platform

The cloud-based platform consists of a comprehensive database and an Application programming interface (API) which may be built up on the standard programming languages for example JavaScript or PHP or HTML or a combination of this programming languages to implement methods for mathematical and probability formula for example the Bayesian inferencing to generate recommendations based on cloud-based database standard data and sensor data.

The data processing algorithm automates the activities of receiving the sensor device data, database standard data, processing the sensor data and standard data; to generate initial matching recommendations and clustering the initial recommendations into a plurality of groups. The method further includes classifying the initial finding candidates using machine learning algorithms integrated into the machine learning algorithm into one or more categories one of the initial finding candidates using type 2 fuzz logic, and determining detection and assessment statistics based on at least the assessed categories and classified findings using Bayesian probability analysis. The method also includes modifying the classified findings and assessed categories based on additional interactive input, and generating the suitable inputs recommendations for example fertilizer rates or soil treatment or seed variety suitability decision based on the determined detection, assessment statistics, and the additional interactive input. Bayesian decision theory is a statistical approach for dataset analysis using pattern classification based on quantifying the trade-offs between various classification decisions using probability.

P(ωj|x)=p(x|ωj)P(ωj)/p(x) Bayes Formula

where

p(x)=Σj p(x|ωj)P(ωj)

Bayes formula shows that by observing the feature vector x, we can convert the prior probability P(ωj) to the posterior probability P(ωj|x)—the probability of the state of nature being ωj given the feature vector values x. The p(x|ωj) is the likelihood of coj with respect to x. For example, if the state coj is being recommended output, p(x|ωj) is the seed variety suitability function of the feature vector x. The other states of co can be being standard crop data on optimum conditions. The recommender engine may be built based on collaborative filtering methods for example k-nearest neighbour (k-NN) approach and Pearson's correlation algorithm to generate precise inferences on the suitable output for example on the suitability of a specific seed variety and quantity of fertilizers based on a combination of ecological data, sensor data, weather data and preferential data from datasets within a particular region and within a group of registered farmers within a specific region.

1. A testing device, comprising: a micro-processor control unit; a memory unit; a display unit; an input unit; a signal conditioning unit; a data encoder unit; a data decoder unit and a communication unit that disseminates interpretable signals in real-time over a communication network.

2. The device recited in claim 1 wherein the communication unit is a cloud based platform comprising a database and an Application Programming Interface (API) which may be built up on programming languages to implement methods for mathematical and probability formula in order to generate recommendations based on test results.

3. The device recited in claim 1 wherein the memory unit is electrically erasable programmable read-only (EEPROM).

4. The device recited in claim 3 wherein the memory unit is expandable.

5. ice recited in claim 2 wherein firmware is stored in the memory unit.

6. The device recited in claim 2 wherein an input and output unit are interfaced on a display unit.

7. The device recited in claim 2 wherein the communication network is Global System for Mobile Communications (GSM).

8. The device recited in claim 1 that is portable and handheld.

9. The device recited in claim 8 that is embedded with on-board power and charging system.
